(19) United States
(12) Patent Application Publication
(10) Pub. No.: US 2023/0220336 A1
(43) Pub. Date: Jul. 13, 2023

(54) FIBROBLASTS AS A REGENERATIVE CELLULAR SOURCE FOR THE TREATMENT OF BLINDNESS

(71) Applicant: FIGENE, LLC, Houston, TX (US)

(72) Inventor: Thomas ICHIM, San Diego, CA (US)

(21) Appl. No.: 17/999,575

(22) PCT Filed: Jun. 1, 2021
(86) PCT No.: PCT/US2021/070649

§ 371 Date: Nov. 22, 2022

Related U.S. Application Data
(60) Provisional application No. 63/033,092, filed on Jun. 1, 2020.

(51) Int. Cl. C12N 5/079; A61K 35/33

(52) U.S. Cl. CPC C12N 5/0621; A61K 35/33; C12N 2500/02; C12N 2501/065; C12N 2502/085; C12N 2506/1307

(57) ABSTRACT

Disclosed are methods and compositions useful for treatment of blindness or dry macular degeneration. In one embodiment, retinal pigmented epithelial cells are generated from fibroblasts through induction of differentiation, and/or transdifferentiation. In another embodiment, fibroblast-derived products, such as differentiated retinal pigmented epithelial cells, are provided to subjects in a therapeutically effective amount.

This application claims priority to U.S. Provisional Patent Application Ser. No. 63/033,092, filed Jun. 1, 2020.

### TECHNICAL FIELD

Embodiments of the disclosure encompass at least the fields of cell biology, molecular biology, and medicine.

### BACKGROUND

For proper human vision, proper function of the retina is needed. The retina is seen as a multi-layered nervous tissue in which energy in the form of light is converted into nerve impulses which reach the brain and provide vision. The layers of the retina include the outermost one, which is closest to the front of the eye, is a layer of neurons that includes ganglion cells. Underneath these ganglion cells is a layer of integrating neurons, and behind the integrating neurons is a layer of photoreceptor cells, called rods and cones. Photoreception in rods and cones begins with absorption of light by a pigment in the cells, the absorbed light causing a receptor potential.

Retinal pigment epithelial (RPE) cells form an intimate structural and functional relationship with the photoreceptor cells in the retinal pigment epithelium, a monolayer of specialized, cuboidal cells located immediately behind the retina. These cells provide support for the photoreceptor cells and carry on important physiological functions, including solute transport, phagocytosis and digestion of discarded outer segments of membranes shed from photoreceptor cells, and drug detoxication.

RPE cells rest on a specialized basement membrane, called the Bruch's membrane, a membrane 1 to 5 microns in thickness and composed of collagen, laminin and other molecules. Underlying the RPE cells is the choriocapillaris of the choroid tissue. The choriocapillaris contains the vasculature to provide nutrients and remove metabolic by-products from the retina. Underlying the choroid tissue is the sclera. It is believed that failure of the RPE cells to properly perform their functions alters the extracellular environment for photoreceptor cells and leads to the eventual degeneration and loss of photoreceptor cells. Dysfunction of RPE contributes to the pathogenesis of a variety of sight-threatening diseases including age-related macular degeneration (ARMD), serious retinal detachment, and such genetic diseases as gyrate atrophy and choroideremia.

Age-related macular degeneration is the leading cause of irreversible visual loss in the developed world. Depending on the definition of the disease, up to 20 million Americans have at least the early stages of AMD [1]. The earliest signs of AMD are known as age-related maculopathy (ARM) and are characterized by the appearance of drusen, subretinal deposits of oxidized lipids and proteins beneath the retinal pigment epithelium as well as variable amounts of visible clumps of pigment in the macula. At this point, the patients are generally asymptomatic with 20/20 vision. In the intermediate stages of AMD, drusen become larger, and pigmentary changes are more severe. In the advanced stages of AMD, patients either develop subretinal neovascularization in which blood vessels from the choriocapillaris break through Bruch's membrane and invade the subretinal or sub-RPE space where they bleed and leak fluid (the exudative or “wet” form of AMD) and ultimately cause permanent loss of macular vision, or else patients develop the advanced “dry” form of AMD in which RPE and photoreceptor cells slowly die leaving behind sharply demarcated regions of dysfunctional macula known as geographic atrophy (GA). Wet AMD accounts for 10-15% of AMD, yet it is responsible for 90% of AMD-related blindness. A recent estimate by the National Eye Institute indicates that 1.75 million Americans have advanced AMD in at least one eye, and about 7.3 million have intermediate AMD and are therefore at high risk for progression to advanced AMD.

One of the first symptoms that patients may notice with AMD progression is distortion of straight lines which can be readily perceived on an Amsler grid, a checkerboard-like chart given to patients for home-testing. Patients are instructed to contact their eye-care provider if new distortion is noted in order to arrange to have a prompt dilated eye examination to look for declines in visual acuity and to assess whether or not there are signs of progression to advanced AMD such as the presence of blood and fluid in the macula or if geographic atrophy is beginning to affect the fovea. If progression is suspected, then imaging tests are generally performed including intravenous angiography with fluorescein or indocyanine green dyes to define the extent of choroidal neovascularization, optical coherence tomography (OCT) to look for macular thickening and pockets of intraretinal, subretinal, and sub-RPE fluid, and autofluorescence imaging to better define the borders of GA and to look for hyperfluorescent borders where GA is likely to progress next.

Generation of RPE cells has been described from embryonic stem cells [2-35] and inducible pluripotent stem cells [36-54], unfortunately both of these cell sources represent a highly undifferentiated phenotype which have the potential to generate tumors or teratomas. Additionally, these cell sources are difficult to generate in large numbers for commercial uses [55].

### BRIEF SUMMARY

The disclosure provides the unexpected discovery that the culturing of fibroblasts in the presence of retinal pigmented epithelial (RPE)-conditioned media allows at least some degree of fibroblast differentiation into RPE cells. Furthermore, the addition of the histone deacetylase inhibitor, valproic acid (as one example), enhances the ability of conditioned media to stimulate differentiation of fibroblasts into RPE cells. In one embodiment, a method of producing differentiated RPE cells comprises: selecting one or more fibroblast cells; introducing the one or more fibroblast cells to a conditioned media, wherein the conditioned media comprises concentrated exosomes derived from fibroblasts and/or a supernatant collected from cultured RPE cells or the progenitors thereof; and culturing the one or more fibroblast cells to produce one or more differentiated RPE cells. In another embodiment, the method further comprises adding to the one or more fibroblast cells one or more agents capable of inducing differentiation. In other embodiments, a method of treating or preventing blindness or macular degeneration in a subject comprises providing to the subject a therapeutically effective amount of fibroblasts or fibroblast-derived products.

Embodiments of the disclosure include methods of producing differentiated retinal pigment epithelial (RPE) cells comprising: selecting one or more fibroblast cells; introducing the one or more fibroblast cells to a conditioned media, wherein the conditioned media comprises concentrated exosomes derived from fibroblasts and/or a supernatant collected from cultured RPE cells or the progenitors thereof; and culturing the one or more fibroblast cells to produce one or more differentiated RPE cells. In specific embodiments, the method further comprises: adding to the one or more fibroblast cells one or more agents capable of inducing differentiation. In specific embodiments, the one or more agents capable of inducing differentiation comprises valproic acid. In some cases, the method further comprises adding hyaluronic acid to the one or more fibroblast cells or subjecting the one or more fibroblast cells to an effective amount of hyaluronic acid.

In some embodiments, the one or more differentiated RPE cells have specific characteristics, such as expressing RPE-65, connexin-8, bestrophin, and/or are capable of phagocytosis.

In specific embodiments, the source of the one or more fibroblast cells is selected from a group consisting of skin; foreskin; hair follicle; adipose; Wharton's Jelly; bone marrow; omentum; placenta; endometrium, and a combination thereof. In specific cases, the conditioned media comprises concentrated exosomes, which may be concentrated by an affinity means, such as an immuno-affinity means. In specific embodiments, the concentrated exosomes express CD6. In some cases, the conditioned media comprises one or more growth factors, such as growth factors selected from the group consisting of CNTF, HGF, interferon gamma, BDNF, neurotrophin, and a combination thereof. The conditioned media may be collected under hypoxic conditions, such as conditions under which hypoxia-inducible factors enter the nucleus at a rate of at least 50% more compared to identical cells cultured in normoxia.

In certain embodiments, following exposure of the cultured RPE cells to one or more inflammatory stimuli, the supernatant is collected. In specific embodiments, the inflammatory stimuli comprises one or more cytokines and/or comprises one or more toll-like receptor agonists.

In particular embodiments, the cultured RPE cells are immortalized. The cultured RPE cells may be primary cells. The cultured RPE cells and the one or more fibroblast cells may have different species of origin. The cultured RPE cells may be of porcine origin. In some cases, the cultured RPE cells are from the cell line ARPE-19.

Embodiments of the disclosure include methods of treating or preventing blindness or macular degeneration in a subject comprising providing to the subject a therapeutically effective amount of fibroblasts and/or fibroblast-derived products. In specific embodiments, the fibroblast-derived products comprise differentiated RPE cells. In some cases, the fibroblast-derived products comprise conditioned media derived from fibroblasts. The fibroblast-derived products may comprise microvesicles, exosomes, apoptotic vesicles, and/or nucleic acids from fibroblasts.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1 is a chart representing the relative mRNA expression levels of the RPE cell marker RPE-65 in samples of fibroblast cells cultured in four different conditions (as the bars read from left to right): control growth media; with RPE-derived supernatant, with valproic acid, and with both RPE-derived supernatant and valproic acid.

FIG. 2 is a chart representing the relative mRNA expression levels of the RPE cell marker CK-8 in samples of fibroblast cells cultured in four different conditions (as the bars read from left to right): control growth media; with RPE-derived supernatant, with valproic acid, and with both RPE-derived supernatant and valproic acid.

FIG. 3 is a chart representing the relative mRNA expression levels of the RPE cell marker bestrophin in samples of fibroblast cells cultured in four different conditions (as the bars read from left to right): control growth media; with RPE-derived supernatant, with valproic acid, and with both RPE-derived supernatant and valproic acid.

### DETAILED DESCRIPTION

### II. Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, “allogeneic” refers to tissues or cells or other material from another body that in a natural setting are immunologically incompatible or capable of being immunologically incompatible, although from one or more individuals of the same species.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, “conditioned medium” describes medium in which a specific cell or population of cells has been cultured for a period of time, and then removed, thus separating the medium from the cell or cells. When cells are cultured in a medium, they may secrete one or more cellular factors, such as factors that can provide trophic support to other cells. Such trophic factors include, but are not limited to hormones, cytokines, extracellular matrix (ECM), proteins, vesicles, antibodies, and/or granules. In this example, the medium comprising the cellular factors is conditioned medium.

As used herein, a “trophic factor” describes a substance that promotes and/or supports survival, growth, proliferation and/or maturation of a cell. Alternatively, or in addition, a trophic factor stimulates increased activity of a cell.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” “treating,” or “therapy” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include cells differentiated from fibroblasts, conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

The term “passaging” refers to the process of transferring a portion of cells from one culture vessel into a new culture vessel.

The term “RPE cell,” as used herein, refers to a retinal pigment epithelial (“RPE”) cell. The term is used generically to refer to RPE cells, regardless of the maturity level of the cells, and thus may encompass RPE cells of varying maturity levels. RPE cells can be visually recognized by their cobblestone-morphology and the initial appearance of pigment. Identification of RPE cells can also be based on the lack of expression of embryonic stem cells markers, such as Oct-4 and Nanog. In addition to the lack of embryonic stem cell markers, RPE cells can also be identified based on the expression of RPE markers, such as RPE-65, PEDF, CRALBP, and/or bestrophin. For example, a cell may be counted as positive for a given marker if the expected staining pattern is observed, e.g., PAX6 localized in the nuclei, bestrophin localized in the plasma membrane in a polygonal pattern (showing localized bestrophin staining in sharp lines at the cell's periphery), ZO-1 staining present in tight junctions outlining the cells in a polygonal pattern, and MITF staining detected confined to the nucleus.

The terms “differentiated RPE cell” and “fibroblast-derived RPE cell” may be used interchangeably throughout to refer broadly to a retinal pigment epithelial (“RPE”) cell differentiated from a pluripotent stem cell, e.g., using the methods disclosed herein. These differentiated RPE cells exhibit the same characteristics as RPE cells, including visual appearance, the expression of RPE markers, and the lack of expression of embryonic stem cell markers.

The terms “mature RPE cell” and “mature differentiated RPE cell,” as used herein, may be used interchangeably throughout to refer broadly to changes that occur following initial differentiating of an RPE cell. Specifically, although RPE cells can be recognized, in part, based on initial appearance of pigment, after differentiation, mature RPE cells can be recognized based on enhanced pigmentation.

### III. Fibroblasts and Cultured Cells

Aspects of the present disclosure comprise cells useful in therapeutic methods and compositions. Cells disclosed herein include, for example, fibroblasts, stem cells (e.g., hematopoietic stem cells or mesenchymal stem cells), and endothelial progenitor cells. Cells of a given type (e.g., fibroblasts) may be used alone or in combination with cells of other types. For example, fibroblasts may be isolated and provided to a subject alone or in combination with one or more stem cells. In some embodiments, disclosed herein are fibroblasts capable of treating or preventing blindness and macular degeneration. In some embodiments, fibroblasts of the present disclosure are adherent to plastic. In some embodiments, the fibroblasts express CD73, CD90, and/or CD105. In some embodiments, the fibroblasts are CD14, CD34, CD45, and/or HLA-DR negative. In some embodiments, the fibroblasts possess the ability to differentiate to osteogenic, chondrogenic, and adipogenic lineage cells.

Compositions of the present disclosure may be obtained from isolated fibroblast cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344, or Stella markers. In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. The cells may be cultured alone or may be cultured in the presence of other cells in order to further upregulate production of growth factors in the conditioned media.

In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-4/5, TrkA, BMP2, BMP4, FGF2, FGF4, PDGF, PGF, TGFα, TGFβ, and/or VEGF.

Fibroblasts may be expanded and utilized by administration themselves or may be cultured in a growth media in order to obtain conditioned media. The term “growth medium” generally refers to a medium sufficient for the culturing of cells, including fibroblasts. In particular, one presently preferred medium for the culturing of the cells herein comprises Dulbecco's Modified Essential Media (DMEM). Another preferred medium for the culturing of the cells herein comprises Ham's F-12 Nutrient Media (F-12). Another particular medium for the culturing of the cells herein comprises Iscove's Modified Dulbecco's Media (IMDM). Particularly preferred is a 1:1 mixture of DMEM and F-12 (DMEM/F-12). The DMEM/F-12 media mixture is preferably supplemented with 15% (v/v) fetal bovine serum (e.g. defined fetal bovine serum, Hyclone™, Logan Utah). In some cases, different growth media are used or different supplementations are provided, and these are normally indicated as supplementations to the growth medium, such as antibiotics/antimycotics (preferably penicillin (100 Units/milliliter), streptomycin (100 milligrams/milliliter), and amphotericin B (0.25 micrograms/milliliter), (Invitrogen*, Carlsbad, Calif.)), and 0.001% (v/v) 2-mercaptoethanol (Sigma®, St. Louis Mo.). Also relating to the present disclosure, the term “standard growth conditions,” as used herein, refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. “Cultured RPE cells,” as used herein, refers to a primary cell culture of RPE cells taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably, these cell numbers are produced within 80, 70, or 60 days or less. In one embodiment, fibroblast cells used are isolated and expanded, and possess one or more markers selected from a group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

When referring to cultured cells, including fibroblast cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors. In some embodiments fibroblasts are transfected with genes to allow for enhanced growth and overcoming of the Hayflick limit.

In some embodiments, the biopsy tissue is washed prior to enzymatic digestion. After washing, a liberase digestive enzyme solution is added without mincing, and the biopsy tissue is incubated at 37.0±0.2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Heidelberg, Germany). After digestion, complete growth media (IMDM, 10% Fetal Bovine Serum (FBS)) is added to neutralize the enzyme. The cells are then pelleted by centrifugation and resuspended in 5.0 mL of complete growth media. Alternatively, centrifugation is not performed, with full inactivation of the enzyme occurring by the addition of the initiation growth media only. Complete growth media is added prior to seeding of the cell suspension into a cell culture flask for initiation of cell growth and expansion. Cells are incubated at 37.0±2.0° C. with 5.0±1.0% CO₂ and fed with fresh complete growth media every three to five days. All feeds in the process are performed by removing half of the complete growth media and replacing the same volume with fresh media. Alternatively, full feeds can be performed. Cells should not remain in the flask greater than 30 days prior to passaging. Confluence is monitored throughout the process to ensure adequate seeding densities during culture splitting. When cell confluence is greater than or equal to 40% in the flask, the cells are passaged by removing the spent media, washing the cells, and treating with Trypsin-EDTA to release adherent cells in the flask into the solution. The cells are then trypsinized and seeded into a larger flask for continued cell expansion. Alternately, one or two smaller flasks, One Layer Cell Stack (1 CS), One Layer Cell Factory (1 CF) or a Two Layer Cell Stack (2 CS) can be used in place of the larger flask. Morphology is evaluated at each passage and prior to harvest to monitor the culture purity throughout the culture purity throughout the process.

In some embodiments, morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37.0±2.0° C. with 5.0±1.0% CO₂ and passaged every three to five days in the larger flask or every five to seven days in a ten layer cell stack (10 CS). Cells should not remain in the flask for more than 10 days prior to passaging. Quality Control (QC) release testing for safety of the Bulk Drug Substance includes sterility and endotoxin testing. When cell confluence in the flask is >95%, cells are passaged to a 10 CS culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional complete growth media is added to neutralize the trypsin, and the cells from the flask are pipetted into a 2-L bottle containing fresh complete growth media. The contents of the 2-L bottle are transferred into the 10 CS and seeded across all layers. Cells are then incubated at 37.-±2.0° C. with 5.0±1.0% CO₂ and fed with fresh complete growth media every five to seven days. Cells should not remain in the 10 CS for more than 20 days prior to passaging. In one embodiment, the passaged dermal fibroblasts are rendered substantially free of immunogenic proteins present in the culture medium by incubating the expanded fibroblasts for a period of time in protein free medium. When cell confluence in the 10 CS is 95% or more, cells are harvested. Harvesting is performed by removing the spent media, washing the cells, treating with trypsin-EDTA to release adherent cells into the solution, and adding additional complete growth media to neutralize the trypsin. Cells are collected by centrifugation, resuspended, and in-process QC tested to determine total viable cell count and cell viability.

The fibroblasts may be fibroblasts obtained from various sources including, for example, dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and fibroblasts obtained from a plastic surgery-related by-product. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-0), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

In some embodiments, fibroblasts are transfected with one or more angiogenic genes to enhance ability to promote angiogenesis. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation sphingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

As disclosed herein, fibroblasts may secret one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts of the present disclosure are cultured with one or more inhibitors of mRNA degradation. In some embodiments, fibroblasts are cultured under conditions suitable to support reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/1, 4.5 g/l, 4 g/l, 3 g/l, 2 g/l or 1 g/l. In some embodiments, such conditions comprise glucose of about 1 g/l.

Aspects of the present disclosure comprise generating conditioned media from fibroblasts. Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500×g). Conditioned media may be filtered through a membrane. The membrane may be a >1000 kDa membrane. Conditioned media may be subject to liquid chromatography such as HPLC. Conditioned media may be separated by size exclusion.

In some embodiments, the present disclosure utilizes exosomes derived from fibroblasts as a therapeutic modality. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/mL and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of, for example, treating or preventing blindness and macular degeneration. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

### IV. Fibroblast Differentiation

In some embodiments, fibroblasts are incubated with one or more growth factors (i.e. mitogenic compounds) under suitable growth conditions, which allows for proliferation and potential differentiation into RPE cells. Likewise, the fibroblasts of the present disclosure may be incubated with one or more various differentiation inducers (i.e. inducers or inducing agents), and optionally one or more growth factors, under suitable conditions to allow for the differentiation and, optionally, propagation of a variety of cell types. As one of ordinary skill would recognize, there are known compounds that function as both growth factors and differentiation inducers. Growth factors useful for proliferation of the fibroblasts include, but are not limited to, M-CSF, IL-6, LIF, and IL-12. Examples of compounds functioning as growth factors and/or differentiation inducers include, but are not limited to, lipopolysaccharide (LPS), phorbol 12-myristate 13-acetate (PMA), stem cell growth factor, human recombinant interleukin-2 (IL-2), IL-3, EGF, b-nerve growth factor (bNGF), recombinant human vascular endothelial growth factor 165 isoform (VEGF165), hepatocyte growth factor (HGF), and hyaluronic acid (HA). Useful doses for inducing proliferation of fibroblasts and increasing susceptibility to differentiation by growth and/or differentiation factors are: 0.5 ng/ml-1.0 μg/ml (preferably 1.0 g/ml) for LPS, 1-160 nM (preferably 3 nM) for PMA, 500-2400 units/ml (preferably 1200 ng/ml) for bNGF, 12.5-100 ng/ml (preferably 50 ng/ml for VEGF), 10-200 ng/ml (preferably 100 ng/ml) for EGF, and 25-200 ng/ml (preferably 50 ng/ml) for HGF.

In one embodiment, inducing differentiation of fibroblast cells into RPE cells is performed by coculturing the fibroblasts with conditioned media derived from cultured RPE cells. In some embodiments, the conditioned media is derived from cultured RPE cells under hypoxic conditions. Hypoxia stimulates the production of growth factors, primarily through the activation of hypoxia-inducible factor 1-alpha (HIF-1α). To activate, HIF-1α enters the nucleus of the cell. In some embodiments, the hypoxic conditions are conditions under which hypoxia-inducible factors enter the nucleus at a rate of at least 50% more compared to identical cells cultured in normoxia. In some embodiments, the media is collected from RPE cells that have been exposed to conditions of less than 21% oxygen for a sufficient period of time to induce the activation of HIF-la.

In one embodiment, hyaluronic acid (HA) is utilized to culture fibroblasts during, and/or subsequent to differentiation into RPE cells. It is known that HA is a major component of the extracellular matrix (ECM) and is particularly prominent in various structures of the eye [56-58]. In some embodiments, HA is added to the growth media at concentrations of 0.01% to 5% by volume. In one embodiment of the disclosure, fibroblasts are utilized to generate RPE cells, with the RPE being differentiated on layers of ECM such as hyaluronic acid in order to allow for improved differentiation into functioning cells.

Another embodiment enhances differentiation into RPE cells by utilizing “dedifferentiating” agents, such as valproic acid, in the presence of fibroblasts and conditioned media from cultured RPE cells.

In some embodiments fibroblasts are first treated with a dedifferentiating agent, such as valproic acid, and/or other agents such as lithium, and/or 5-azacytidine in order to induce expression of one or more markers, which may comprise OCT-4, alkaline phosphatase, Sox2, TDGF-1, SSEA-3, SSEA-4, TRA-1-60, and/or TRA-1-80. The dedifferentiated cells may be cultured in a multilayer population or embryoid body for a time sufficient for pigmented epithelial cells to appear in said culture. The time sufficient for pigmented epithelial cells to appear in the culture may comprise at least about 1 week, at least about 2 weeks, at least about 3 weeks, at least about 4 weeks, at least about 5 weeks, at least about 6 weeks, or at least about 7 weeks, at least about 8 weeks. The multilayer population or embryoid body may be cultured in a medium that comprises DMEM or DMEM/F-16. The medium may comprise embryoid body differentiation medium (EB-DM). The pigmented epithelial cells may be isolated and cultured, thereby producing a population of RPE cells. The isolating may comprise dissociating cells or clumps of cells from the culture enzymatically, chemically, or physically and selecting pigmented epithelial cells or clumps of cells may comprise pigmented epithelial cells. The embryoid body may be cultured in suspension and/or as an adherent culture (e.g., in suspension followed by adherent culture). The embryoid body cultured as an adherent culture may produce one or more outgrowths comprising pigmented epithelial cells. In some embodiments, the pluripotent stem cells have reduced HLA antigen complexity. In other embodiments, prior to RPE formation, the dedifferentiated fibroblasts cells may be cultured on a matrix which may be selected from the group consisting of laminin, fibronectin, vitronectin, proteoglycan, entactin, collagen, collagen I, collagen IV, collagen VIII, heparan sulfate, Matrigel™ (a soluble preparation from Engelbreth-Holm-Swarm (EHS) mouse sarcoma cells), CellStart, a human basement membrane extract, or any combination thereof.

In some embodiments, RPE cells express one or more RPE cell markers. The one or more RPE cell markers may comprise RPE65, CRALBP, PEDF, bestrophin, MITF, Otx2, PAX2, PAX6, ZO-1, and/or tyrosinase. The RPE cells may be produced by a method comprising maintaining RPE cells as quiescent cells for a time sufficient to attain an average melanin content. The RPE cells may be produced by a method comprising maintaining RPE cells as quiescent cells for a time sufficient to establish bestrophin expression in at least 50% of the RPE cells. The RPE may be produced by a method comprising culturing the RPE cells under conditions that increase expression of one or more alpha integrin subunit, e.g., alpha integrin subunit 1, alpha integrin subunit 2, alpha integrin subunit 3, alpha integrin subunit 4, alpha integrin subunit 5, alpha integrin subunit 6, or alpha integrin subunit 9. The conditions may comprise exposure to manganese, exposure to an anti-CD29 antibody, exposure to monoclonal antibody HUTS-21, exposure to monoclonal antibody mAb TS2/16, and/or passaging said RPE cells for at least about 4 passages.

### V. Administration of Therapeutic Compositions

The therapy provided herein may comprise administration of a therapeutic agents (e.g., fibroblasts, exosomes from fibroblasts, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts) of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

In another embodiment, a pharmaceutical preparation comprising fibroblast-derived RPE cells suitable for treatment of blindness and macular degeneration, wherein said fibroblast-derived RPE cells contain an average melanin content of less than 8 pg/cell, and wherein said RPE cells may have at least one of the following properties: a consistent phenotype after transplantation for at least about one month; a consistent phenotype in culture for at least about one month; integration into the host after transplantation; no proliferation after transplantation; undergo phagocytosis; deliver, metabolize, or store vitamin A; transportation of iron between the retina and choroid after transplantation; attachment to the Bruch's membrane after transplantation; absorbing stray light after transplantation; an elevated expression of alpha integrin subunits; a greater average telomere length than RPE cells derived from donated human tissue; a greater replicative lifespan in culture than RPE cells derived from donated human tissue; a greater expression of one or more alpha integrin subunits than RPE cells derived from donated human tissue; a lower A2E content than RPE cells derived from donated human tissue; a lower lipofuscin content than RPE cells derived from donated human tissue; less accumulated ultraviolet damage than RPE cells derived from donated human tissue; or a greater number of phagosomes than RPE cells derived from donated human tissue. In one aspect, the present disclosure provides a pharmaceutical preparation comprising RPE cells suitable for the treatment of blindness and retinal degeneration, wherein the RPE cells contain an average melanin content of less than 8 pg/cell and have at least one of the following properties: attachment to the Bruch's membrane after transplantation; absorption of stray light after transplantation; a greater average telomere length than RPE cells derived from donated human tissue; a greater replicative lifespan in culture than RPE cells derived from donated human tissue; a lower A2E content than RPE cells derived from donated human tissue; a lower lipofuscin content than RPE cells derived from donated human tissue; less accumulated ultraviolet damage than RPE cells derived from donated human tissue; or a greater number of phagosomes than RPE cells derived from donated human tissue.

In some embodiments, fibroblasts are utilized to protect an autologous or allogeneic retinal transplant from rejection.

### VI. Kits of the Disclosure

Any of the cellular and/or non-cellular compositions described herein or similar thereto may be comprised in a kit. In a non-limiting example, one or more reagents for use in methods for preparing fibroblasts or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### EXAMPLES

The following example is included to demonstrate a particular embodiment of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Differentiation of Fibroblasts into RPE Cells

In this example, four different samples of fibroblasts were cultured: (1) fibroblasts cultured in complete growth media (control); (2) fibroblasts cultured in conditioned media from cultured ARPE-19 cells (supernatant only); (3) fibroblasts cultured in complete growth media with valproic acid (valproic acid only); and 4) fibroblasts cultured in conditioned media from cultured ARPE-19 cells with valproic acid (supernatant+valproic acid).

Preparation of conditioned media from cultured RPE cells. ARPE-19 cells were purchased from ATCC (catalogue CRL-2302™) and grown in complete growth media (DMEM/F-12 media, 10% fetal bovine serum), passaged according to the manufacturer's instructions. Corning® T-75 flasks (catalog #430641) were used for subculturing. The media was removed and discarded. The remaining cell layer was subsequently rinsed with 0.05% (w/v) Trypsin-0.53 mM EDTA solution to remove all traces of serum that may contain trypsin inhibitor. Next, 2.0 to 3.0 mL of Trypsin-EDTA solution was added to the flask, and the cells were observed under an inverted microscope until the cell layers dispersed (usually within 5 to 15 minutes). While waiting for the cells to detach, care was taken to avoid agitation of the cells in the flask in order to minimize clumping. Cells that did not detach were placed at 37° C. to facilitate dispersal. After the cells dispersed, 6.0 to 8.0 mL of complete growth medium was added to the flask, and the cells were aspirated by gently pipetting. In order to remove the trypsin-EDTA solution, the cell suspension was transferred to a centrifuge tube and spun at approximately 125 g for 5 to 10 minutes. The supernatant was discarded, and the cells were resuspended in fresh growth medium. Conditioned media from the cultured ARPE-19 cells was generated at passage 5 to 7, centrifuged to remove particulate matter at 700 g for 15 minutes, and then filter sterilized with a 0.2 micron filter.

Preparation and culturing of fibroblast cells. All four samples evaluated foreskin fibroblast cells (ATCC). Fibroblast cells were plated in complete growth media ((DMEM/F-12 media, 10% fetal bovine serum) and incubated at 37° C. in a fully humidified incubator with 5% CO₂. For the fibroblast samples cultured in the presence of valproic acid, a solution of valproic acid was added to the growth media at a concentration of 1 μg/mL. For samples cultured in conditioned media, the conditioned media was added to the fibroblast culture at a ratio of 1 to 8 volume by volume, respectively. Following 0, 7, and 14 days of culturing, the mRNA expression levels of RPE markers in the cultured cells were analyzed by RT-PCR.

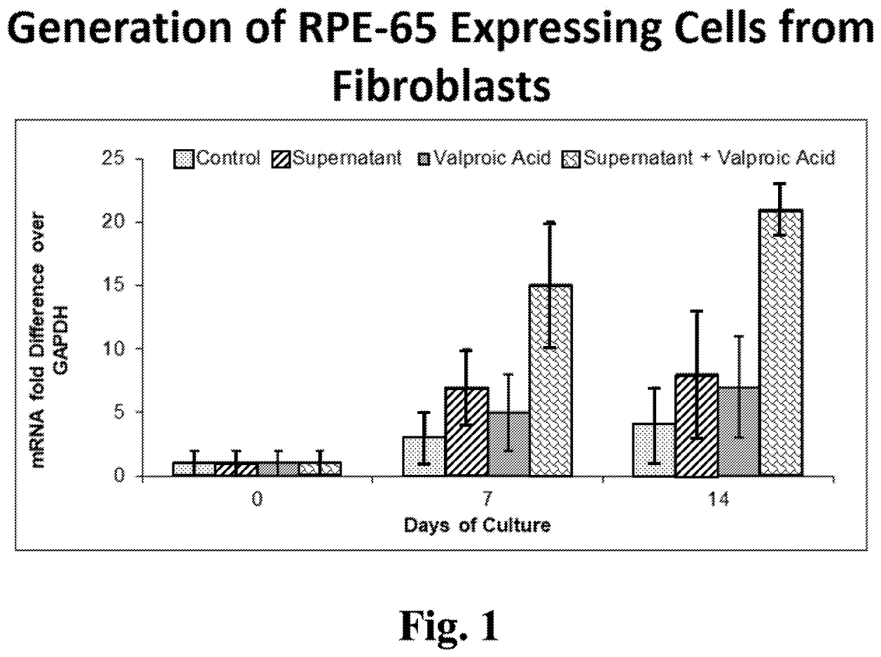

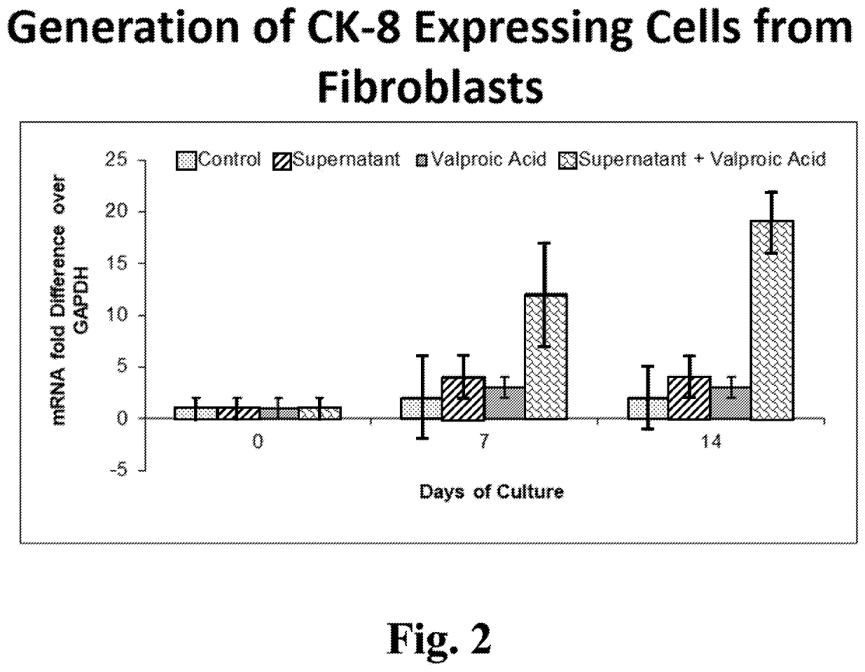

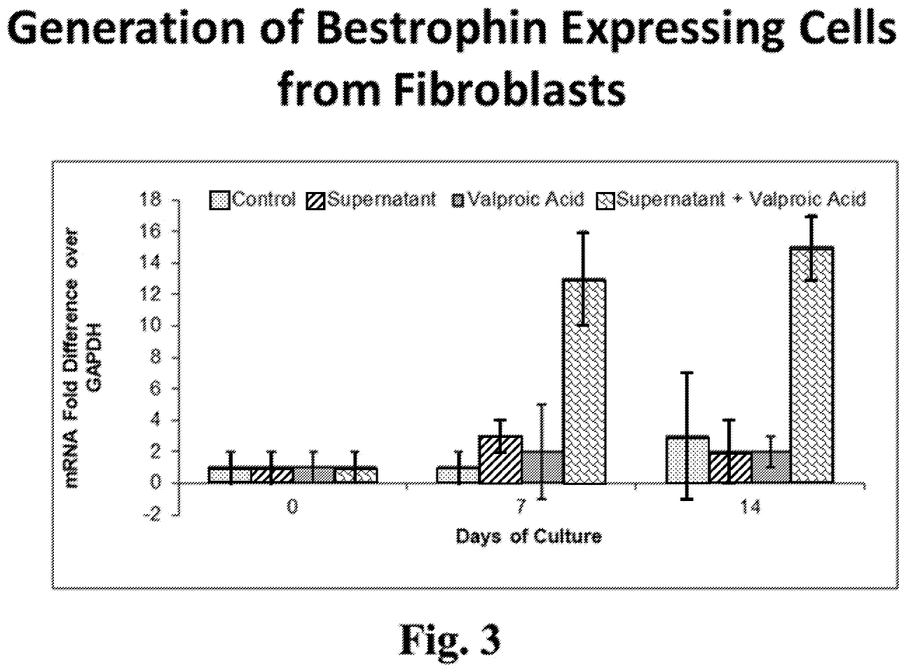

FIGS. 1-3 show the mRNA expression levels of three RPE markers, RPE-65, CK-8, and bestrophin, respectively, in the four different fibroblast cell cultures after 0, 7, and 14 days of culturing. The mRNA values are represented as a ratio of RPE marker expression to GAPDH expression. The fibroblast cells cultured with a combination of valproic acid and conditioned media showed significant increases in the expression of all three RPE markers, highlighting the differentiation of the cells into RPE cells.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Bernstein, P. S., Nutritional Interventions against Age-Related Macular Degeneration. Acta Hortic, 2009. 841: p. 103-112.
2. Osakada, F., et al., Toward the generation of rod and cone photoreceptors from mouse, monkey and human embryonic stem cells. Nat Biotechnol, 2008. 26(2): p. 215-24.
3. Gong, J., et al., Effects of extracellular matrix and neighboring cells on induction of human embryonic stem cells into retinal or retinal pigment epithelial progenitors. Exp Eye Res, 2008. 86(6): p. 957-65.
4. Carr, A. J., et al., Molecular characterization and functional analysis of phagocytosis by human embryonic stem cell-derived RPE cells using a novel human retinal assay. Mol Vis, 2009. 15: p. 283-95.
5. Idelson, M., et al., Directed differentiation of human embryonic stem cells into functional retinal pigment epithelium cells. Cell Stem Cell, 2009. 5(4): p. 396-408.
6. Wang, N. K., et al., Transplantation of reprogrammed embryonic stem cells improves visual function in a mouse model for retinitis pigmentosa. Transplantation, 2010. 89(8): p. 911-9.
7. Juuti-Uusitalo, K., et al., Efflux protein expression in human stem cell-derived retinal pigment epithelial cells. PLoS One, 2012. 7(1): p. e30089.
8. Huang, J., et al., Embryonic stem-cell-derived retinal pigment epithelial cells for macular degeneration. Lancet, 2012. 379(9831): p. 2050; author reply 2050-1.
9. Zhang, Y. S., et al., Derivation, culture and retinal pigment epithelial differentiation of human embryonic stem cells using human fibroblast feeder cells. J Assist Reprod Genet, 2012. 29(8): p. 735-44.
10. Cho, M. S., et al., Generation of retinal pigment epithelial cells from human embryonic stem cell-derived spherical neural masses. Stem Cell Res, 2012. 9(2): p. 101-9.
11. Jin, Z. B. and M. Takahashi, Generation of retinal cells from pluripotent stem cells. Prog Brain Res, 2012. 201: p. 171-81.
12. Reynolds, J. and D. A. Lamba, Human embryonic stem cell applications for retinal degenerations. Exp Eye Res, 2014. 123: p. 151-60.
13. Sorkio, A., et al., Structure and barrier properties of human embryonic stem cell-derived retinal pigment epithelial cells are affected by extracellular matrix protein coating.

Tissue Eng Part A, 2014. 20(3-4): p. 622-34.
14. Hsiung, J., D. Zhu, and D. R. Hinton, Polarized human embryonic stem cell-derived retinal pigment epithelial cell monolayers have higher resistance to oxidative stress-induced cell death than nonpolarized cultures. Stem Cells Transl Med, 2015. 4(1): p. 10-20.
15. Sowden, J. C., ESC-derived retinal pigmented epithelial cell transplants in patients: so far, so good. Cell Stem Cell, 2014. 15(5): p. 537-8.
16. Pennington, B. O., et al., Defined culture of human embryonic stem cells and xeno-free derivation of retinal pigmented epithelial cells on a novel, synthetic substrate. Stem Cells Transl Med, 2015. 4(2): p. 165-77.
17. Lidgerwood, G. E., et al., Defined Medium Conditions for the Induction and Expansion of Human Pluripotent Stem Cell-Derived Retinal Pigment Epithelium. Stem Cell Rev Rep, 2016. 12(2): p. 179-88.
18. Ilmarinen, T., et al., Ultrathin Polyimide Membrane as Cell Carrier for Subretinal Transplantation of Human Embryonic Stem Cell Derived Retinal Pigment Epithelium. PLoS One, 2015. 10(11): p. e0143669.
19. Hu, J., T. C. He, and F. Li, For your eyes only: Harnessing human embryonic stem cell-derived retinal pigment epithelial cells to improve impaired vision. Genes Dis, 2015. 2(4): p. 293-294.
20. Shim, S. H., et al., Survival of Transplanted Human Embryonic Stem Cell-Derived Retinal Pigment Epithelial Cells in a Human Recipient for 22 Months. JAMA Ophthalmol, 2017. 135(3): p. 287-289.
21. Petrus-Reurer, S., et al., Integration of Subretinal Suspension Transplants of Human Embryonic Stem Cell-Derived Retinal Pigment Epithelial Cells in a Large-Eyed Model of Geographic Atrophy. Invest Ophthalmol Vis Sci, 2017. 58(2): p. 1314-1322.
22. Bennis, A., et al., Stem Cell Derived Retinal Pigment Epithelium: The Role of Pigmentation as Maturation Marker and Gene Expression Profile Comparison with Human Endogenous Retinal Pigment Epithelium. Stem Cell Rev Rep, 2017. 13(5): p. 659-669.
23. Skottman, H., et al., Contacting co-culture of human retinal microvascular endothelial cells alters barrier function of human embryonic stem cell derived retinal pigment epithelial cells. Exp Cell Res, 2017. 359(1): p. 101-111.
24. Foltz, L. P. and D. O. Clegg, Rapid, Directed Differentiation of Retinal Pigment Epithelial Cells from Human Embryonic or Induced Pluripotent Stem Cells. J Vis Exp, 2017(128).
25. Ben M'Barek, K., et al., Human ESC-derived retinal epithelial cell sheets potentiate rescue of photoreceptor cell loss in rats with retinal degeneration. Sci Transl Med, 2017. 9(421).
26. Behroozi, F., et al., Smart liposomal drug delivery for treatment of oxidative stress model in human embryonic stem cell-derived retinal pigment epithelial cells. Int J Pharm, 2018. 548(1): p. 62-72.
27. Abu Khamidakh, A. E., et al., Wound healing of human embryonic stem cell-derived retinal pigment epithelial cells is affected by maturation stage. Biomed Eng Online, 2018. 17(1): p. 102.
28. Idelson, M., et al., Immunological Properties of Human Embryonic Stem Cell-Derived Retinal Pigment Epithelial Cells. Stem Cell Reports, 2018. 11(3): p. 681-695.
29. Karamali, F., et al., Hepatocyte growth factor promotes the proliferation of human embryonic stem cell derived retinal pigment epithelial cells. J Cell Physiol, 2019. 234(4): p. 4256-4266.
30. Liu, Y., et al., Human embryonic stem cell-derived retinal pigment epithelium transplants as a potential treatment for wet age-related macular degeneration. Cell Discov, 2018. 4: p. 50.
31. Kharitonov, A. E., et al., Possibilities for Using Pluripotent Stem Cells for Restoring Damaged Eye Retinal Pigment Epithelium. Acta Naturae, 2018. 10(3): p. 30-39.
32. Ilmarinen, T., et al., Survival and functionality of xeno-free human embryonic stem cell-derived retinal pigment epithelial cells on polyester substrate after transplantation in rabbits. Acta Ophthalmol, 2019. 97(5): p. e688-e699.
33. Artero-Castro, A., et al., The identification of small molecules that stimulate retinal pigment epithelial cells: potential novel therapeutic options for treating retinopathies. Expert Opin Drug Discov, 2019. 14(2): p. 169-177.
34. Szatmari-Toth, M., et al., Human Embryonic Stem Cell-Derived Retinal Pigment Epithelium-Role in Dead Cell Clearance and Inflammation. Int J Mol Sci, 2019. 20(4).
35. Qiu, T. G., Transplantation of human embryonic stem cell-derived retinal pigment epithelial cells (MA09-hRPE) in macular degeneration. NPJ Regen Med, 2019. 4: p. 19.
36. Carr, A. J., et al., Protective effects of human iPS-derived retinal pigment epithelium cell transplantation in the retinal dystrophic rat. PLoS One, 2009. 4(12): p. e8152.
37. Hu, Q., et al., Memory in induced pluripotent stem cells: reprogrammed human retinal-pigmented epithelial cells show tendency for spontaneous redifferentiation. Stem Cells, 2010. 28(11): p. 1981-91.
38. Okamoto, S. and M. Takahashi, Induction of retinal pigment epithelial cells from monkey iPS cells. Invest Ophthalmol Vis Sci, 2011. 52(12): p. 8785-90.
39. Krohne, T. U., et al., Generation of retinal pigment epithelial cells from small molecules and OCT4 reprogrammed human induced pluripotent stem cells. Stem Cells Transl Med, 2012. 1(2): p. 96-109.
40. Kuroda, T., et al., Highly sensitive in vitro methods for detection of residual undifferentiated cells in retinal pigment epithelial cells derived from human iPS cells. PLoS One, 2012. 7(5): p. e37342.
41. Mekala, S. R., et al., Derivation, characterization and retinal differentiation of induced pluripotent stem cells. J Biosci, 2013. 38(1): p. 123-34.
42. Maeda, T., et al., Retinal pigmented epithelial cells obtained from human induced pluripotent stem cells possess functional visual cycle enzymes in vitro and in vivo. J Biol Chem, 2013. 288(48): p. 34484-93.
43. Ferrer, M., et al., A multiplex high-throughput gene expression assay to simultaneously detect disease and functional markers in induced pluripotent stem cell-derived retinal pigment epithelium. Stem Cells Transl Med, 2014. 3(8): p. 911-22.
44. Reichman, S., et al., From confluent human iPS cells to self-forming neural retina and retinal pigmented epithelium. Proc Natl Acad Sci USA, 2014. 111(23): p. 8518-23.
45. Hansson, M. L., et al., Efficient delivery and functional expression of transfected modified mRNA in human embryonic stem cell-derived retinal pigmented epithelial cells. J Biol Chem, 2015. 290(9): p. 5661-72.
46. Sugita, S., et al., Inhibition of T-cell activation by retinal pigment epithelial cells derived from induced pluripotent stem cells. Invest Ophthalmol Vis Sci, 2015. 56(2): p. 1051-62.
47. Sun, J., et al., Protective Effects of Human iPS-Derived Retinal Pigmented Epithelial Cells in Comparison with Human Mesenchymal Stromal Cells and Human Neural Stem Cells on the Degenerating Retina in rdl mice. Stem Cells, 2015. 33(5): p. 1543-53.
48. Sugita, S., et al., Lack of T Cell Response to iPSC-Derived Retinal Pigment Epithelial Cells from HLA Homozygous Donors. Stem Cell Reports, 2016. 7(4): p. 619-634.
49. Greene, W. A., et al., Secretion Profile of Induced Pluripotent Stem Cell-Derived Retinal Pigment Epithelium During Wound Healing. Invest Ophthalmol Vis Sci, 2016. 57(10): p. 4428-4441.
50. Reichman, S., et al., Generation of Storable Retinal Organoids and Retinal Pigmented Epithelium from Adherent Human iPS Cells in Xeno-Free and Feeder-Free Conditions. Stem Cells, 2017. 35(5): p. 1176-1188.
51. Mandai, M., et al., Autologous Induced Stem-Cell-Derived Retinal Cells for Macular Degeneration. N Engl J Med, 2017. 376(11): p. 1038-1046.
52. Hazim, R. A., et al., Differentiation of RPE cells from integration-free iPS cells and their cell biological characterization. Stem Cell Res Ther, 2017. 8(1): p. 217.
53. Fanelli, G., et al., Human stem cell-derived retinal epithelial cells activate complement via collectin 11 in response to stress. Sci Rep, 2017. 7(1): p. 14625.
54. Matsumoto, E., et al., Fabricating retinal pigment epithelial cell sheets derived from human induced pluripotent stem cells in an automated closed culture system for regenerative medicine. PLoS One, 2019. 14(3): p. e0212369.
55. Mehat, M. S., et al., Transplantation of Human Embryonic Stem Cell-Derived Retinal Pigment Epithelial Cells in Macular Degeneration. Ophthalmology, 2018. 125(11): p. 1765-1775.
56. Yang, I. H., et al., Macrophages promote a profibrotic phenotype in orbital fibroblasts through increased hyaluronic acid production and cell contractility. Sci Rep, 2019. 9(1): p. 9622.
57. Suarez-Barrio, C., et al., Hyaluronic Acid Combined with Serum Rich in Growth Factors in Corneal Epithelial Defects. Int J Mol Sci, 2019. 20(7).
58. Sun, M., et al., Hyaluronan Derived From the Limbus is a Key Regulator of Corneal Lymphangiogenesis. Invest Ophthalmol Vis Sci, 2019. 60(4): p. 1050-1062.

What is claimed is:

1. A method of producing differentiated retinal pigment epithelial (RPE) cells, comprising:
introducing one or more fibroblast cells to a conditioned media, wherein the conditioned media comprises concentrated exosomes derived from fibroblasts and/or a supernatant collected from cultured RPE cells or the progenitors thereof; and
culturing the one or more fibroblast cells to produce one or more differentiated RPE cells.

2. The method of claim 1, further comprising:
adding to the one or more fibroblast cells one or more agents capable of inducing differentiation.

3. The method of claim 2, wherein the one or more agents capable of inducing differentiation comprises valproic acid.

4. The method of claims 2-3, further comprising:
adding hyaluronic acid to the one or more fibroblast cells.

5. The method of one of claims 1-4, wherein the one or more differentiated RPE or cells express RPE-65.

6. The method of one of claims 1-4, wherein the one or more differentiated RPE cells express connexin-8.

7. The method of one of claims 1-4, wherein the one or more differentiated RPE cells express bestrophin.

8. The method of one of claims 1-7, wherein the one or more differentiated RPE cells are capable of phagocytosis.

9. The method of one of claims 1-8, wherein the one or more fibroblast cells' source is selected from a group consisting of: skin; foreskin; hair follicle; adipose; Wharton's Jelly; bone marrow; omentum; placenta; and endometrium.

10. The method of one of claims 1-9, wherein the conditioned media comprises concentrated exosomes.

11. The method of one of claims 1-9, wherein the concentrated exosomes are concentrated by an affinity means.

12. The method of one of claims 1-9, wherein the concentrated exosomes are concentrated by an immuno-affinity means.

13. The method of one of claims 10-12, wherein the concentrated exosomes express CD6.

14. The method of one of claims 1-13, wherein the conditioned media comprises one or more growth factors.

15. The method of claim 14, wherein the one or more growth factors comprises growth factors selected from the group consisting of CNTF, HGF, interferon gamma, BDNF, and neurotrophin.

16. The method of one of claims 1-15, wherein the conditioned media is collected under hypoxic conditions.

17. The method of claim 16, wherein the hypoxic conditions are conditions under which hypoxia-inducible factors enter the nucleus at a rate of at least 50% more compared to identical cells cultured in normoxia.

18. The method of one of claims 1-17, wherein the supernatant is collected after exposure of the cultured RPE cells to an inflammatory stimuli.

19. The method of claim 18, wherein the inflammatory stimuli is a cytokine.

20. The method of claim 18, wherein the inflammatory stimuli is a toll-like receptor agonist.

21. The method of one of claims 1-20, wherein the cultured RPE cells are immortalized.

22. The method of one of claims 1-20, wherein the cultured RPE cells are primary cells.

23. The method of one of claims 1-22, wherein the cultured RPE cells and the one or more fibroblast cells have different species of origin.

24. The method of claim 23, wherein the cultured RPE cells are of porcine origin.

25. The method of claim one of claims 1-23, wherein the cultured RPE cells are the cell line ARPE-19.

26. A method of treating or preventing blindness or macular degeneration in a subject comprising providing to the subject a therapeutically effective amount of fibroblasts or fibroblast-derived products.

27. The method of claim 26, wherein the method comprises providing to the subject an effective amount of fibroblasts.

28. The method of claim 26, wherein the method comprises providing to the subject an effective amount of fibroblast-derived products.

29. The method of claim 28, wherein the fibroblast-derived products comprise differentiated RPE cells.

30. The method of claim 28, wherein the fibroblast-derived products comprise conditioned media derived from fibroblasts.

31. The method of claim 28, wherein the fibroblast-derived products comprise microvesicles from fibroblasts.

32. The method of claim 28, wherein the fibroblast-derived products comprise exosomes from fibroblasts.

33. The method of claim 28, wherein the fibroblast-derived products comprise apoptotic vesicles from fibroblasts.

34. The method of claim 28, wherein the fibroblast-derived products comprise nucleic acids from fibroblasts.
